(19) United States
(12) Patent Application Publication
(10) Pub. No.: US 2023/0172884 A1
(43) Pub. Date: Jun. 8, 2023

(54) USE OF BUCILLAMINE IN THE TREATMENT OF INFECTIOUS DISEASES

(71) Applicant: Revive Therapeutics Ltd, Toronto (CA)

(72) Inventors: Fabio Andrea Chianelli, Toronto (CA); Michael Frank, Toronto (CA)

(73) Assignee: Revive Therapeutics Ltd, Toronto, ON (CA)

(21) Appl. No.: 17/912,597

(22) PCT Filed: Mar. 16, 2021
(86) PCT No.: PCT/CA2021/050350

§ 371 Date: Sep. 19, 2022

Related U.S. Application Data
(60) Provisional application No. 62/991,996, filed on Mar. 19, 2020.

(51) Int. Cl. A61K 31/198; A61P 31/14

(52) U.S. Cl. CPC A61K 31/198; A61P 31/14

(57) ABSTRACT

Methods and uses for the treatment or prevention of an infectious disease in a mammal including in a human, including administering a therapeutically effective amount of bucillamine or a pharmaceutically acceptable salt or solvate thereof to a mammal in need thereof. The infectious disease can include influenza. The infectious disease can also include coronavirus disease 2019.

### FIELD

The present invention relates to pharmaceutical compositions comprising bucillamine and their use for the treatment of infectious diseases.

### BACKGROUND

Current antiviral interventions for influenza have exhibited modest efficacy, especially in improving mortality in at-risk populations, such as the elderly.^{1,2} Novel antivirals have been plagued by poor oral bioavailability and lack of efficacy when not delivered early.¹ This is because these drugs mostly act to prevent the early processes of virus binding to cells or viral replication.² Thiols, particularly N-acetylcysteine (NAC), with antioxidant and reducing activity have been investigated as effective therapies that abrogate the potential for influenza to cause severe disease.^{3,4,5} Restoration of glutathione, the major intracellular thiol antioxidant, is a critical functional activity of NAC.⁶ Reactive oxygen species (ROS) generation during influenza virus infection aggravate destructive inflammation and programmed death of epithelial cells.⁷ Studies in human cells and animal models have shown that NAC works to prevent acute lung injury caused by influenza virus infection through inhibition of these ROS-mediated mechanisms.^{4,7} NAC has been investigated clinically and found to significantly attenuate clinical symptoms associated with influenza infection, especially in elderly at-risk patients.⁵ While NAC is easily taken up by cells and has low toxicity, clinical efficacy has required long-term and high-dose administration because of modest relative potency, limiting its clinical applicability.

When an immune response is triggered by an Influenza virus, this results in the accumulation of cells which secrete a variety of toxic chemicals, such as reactive oxygen species. These chemicals are intended to kill infected cells, but they can also damage surrounding healthy tissue and themselves cause some of the symptoms associated with Influenza.

Any discussion of documents, acts, materials, devices, articles or the like which has been included in the present specification is not to be taken as an admission that any or all of these matters form part of the prior art base or were common general knowledge in the field relevant to the present invention as it existed before the priority date of each claim of this application.

### SUMMARY OF THE INVENTION

Bucillamine (N-(mercapto-2-methylpropionyl)-l-cysteine) is a cysteine derivative with 2 thiol groups. The structure of bucillamine is:

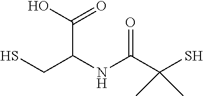

This compound has also been shown to prevent oxidative and reperfusion injury in heart and liver tissues.⁸ Bucillamine is non-toxic with proven safety and activity as a clinically approved IND therapeutic intervention for cystinuria and rheumatoid arthritis and is highly cell permeable for efficient delivery into cells.^{8,9} Bucillamine is 16-fold more potent than NAC as a thiol donor in vivo, giving it vastly superior function in restoring glutathione.

Bucillamine provides protection against oxidative stress by donating thiol groups to GSH. This reactivates GSH and increases the amount available to remove reactive oxygen species, enhancing its endogenous antioxidant activity. The present inventors hypthesized that Bucillamine can be used to treat infectious disease symptoms, such as influenza symptoms and coronavirus disease 2019 (COVID 19) symptoms, by reducing tissue damage from reactive oxygen species.

The present disclosure, in one aspect, relates to a method for the treatment of an infectious disease in a mammal comprising administering a therapeutically effective amount of bucillamine or a pharmaceutically acceptable salt or solvate thereof, to a mammal in need thereof. In one aspect, the infectious disease is influenza. In another aspect, the infectious disease is coronavirus disease 2019. In a further aspect, the method of treatment is for preventing or reducing acute lung injury during an infection. In certain aspects of methods of the present disclosure, pharmaceutically acceptable compositions of the present disclosure can be administered to humans and other animals at a unit dose within range of about 10 mg to about 50 mg, the range of 100 mg to about 200 mg, 100 mg, and 200 mg and this should provide a therapeutically effective dose. In another aspect, the daily dose is 300 mg per day. In another aspect, the daily does is 600 mg per day. In certain aspects, the unit dose may be higher than 200 mg. In another aspect, the daily dose is 600 mg per day. In certain aspects, the unit dose may be 200 mg to 300 mg. In certain other aspects, the daily dose may be higher than 600 mg per day. In another aspect, the daily dose is 600 mg to 800 mg per day. In another aspect, the daily dose is up to 1000 mg per day. In other aspects, the daily dose is up to 1500 mg per day, up to 2000 mg per day, up to 2500 mg per day, up to 3000 mg per day, 300-600 mg per day, 300-1000 mg per day, 300-1500 mg per day, 300-2000 mg per day, 300-2500 mg per day, 300-3000 mg per day, 600-1000 mg per day, 600-1500 mg per day, 600-2000 mg per day, 600-2500 mg per day, 600-3000 mg per day. However, the daily dose will necessarily be varied depending upon the host treated, the particular route of administration, and the severity of the illness being treated. Accordingly, the optimum dosage may be determined by the practitioner who is treating any particular patient.

The present disclosure, in another aspect, relates to a use of a pharmaceutical composition including bucillamine or a pharmaceutically acceptable salt or solvate thereof, together with one or more pharmaceutically acceptable carriers, diluents and excipients for the treatment of an infectious disease. In another aspect, the infectious disease is influenza. In another aspect, the infectious disease is coronavirus disease 2019. In another aspect, the use is for preventing or reducing acute lung injury during an infection. In certain aspects of uses of the present disclosure, pharmaceutically acceptable compositions of the present disclosure can be used at a unit dose within range of about 10 mg to about 50 mg, the range of 100 mg to about 200 mg, 100 mg, and 200 mg and this should provide a therapeutically effective dose. However, the daily dose will necessarily be varied depending upon the host treated, the particular route of administration, and the severity of the illness being treated. Accordingly, the optimum dosage may be determined by the practitioner who is treating any particular patient.

### DETAILED DESCRIPTION

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

For use in therapy a therapeutically effective amount of the bucillamine or pharmaceutically acceptable salts or solvates thereof, may be presented as a pharmaceutical composition. Thus, in a further embodiment the invention provides a pharmaceutical composition of bucillamine or pharmaceutically acceptable salts or solvates thereof in admixture with one or more pharmaceutically acceptable carriers, diluents, or excipients. The carrier(s), diluent(s) or excipient(s) must be acceptable in the sense of being compatible with the other ingredients of the formulation and not deleterious to the recipient thereof.

When applicable, the compositions of the present invention, including bucillamine may be in the form of and/or may be administered as a pharmaceutically acceptable salt.

Typically, a pharmaceutically acceptable salt may be readily prepared by using a desired acid or base as appropriate. The salt may precipitate from solution and be collected by filtration or may be recovered by evaporation of the solvent.

Suitable addition salts are formed from acids which form non-toxic salts and examples are hydrochloride, hydrobromide, hydroiodide, sulphate, nitrate, phosphate, hydrogen phosphate, dihydrogen phosphate acetate, maleate, malate, fumarate, lactate, tartrate, citrate, formate, gluconate, succinate, pyruvate, oxalate, oxaloacetate, trifluoroacetate, saccharinate, benzoate, methanesulphonate, ethanesulphonate, benzenesulphonate, p-toluenesulphonate and isethionate.

Suitable salts may also be formed from bases, forming salts including ammonium salts, alkali metal salts such as those of sodium and potassium, alkaline earth metal salts such as those of calcium and magnesium.

Pharmaceutically acceptable salts may also be prepared from other salts, including other pharmaceutically acceptable salts, using conventional methods.

Those skilled in the art of organic or coordination chemistry will appreciate that many organic and coordination compounds can form complexes with solvents in which they are reacted or from which they are precipitated or crystallized. These complexes are known as “solvates”. For example, a complex with water is known as a “hydrate”. Solvates of bucillamine are within the scope of the present invention.

Pharmaceutical compositions of the invention may be formulated for administration by any appropriate route, for example by the oral (including buccal or sublingual). Therefore, the pharmaceutical compositions of the invention may be formulated, for example, as tablets, capsules, powders, granules, lozenges, creams or liquid preparations, such as oral solutions or suspensions. Such pharmaceutical formulations may be prepared by any method known in the art of pharmacy, for example by bringing into association the active ingredient with the carrier(s) or excipient(s).

Tablets and capsules for oral administration may be in unit dose presentation form, and may contain conventional excipients such as binding agents, for example syrup, acacia, gelatine, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatine, hydroxyethyl cellulose, carboxymethyl cellulose, aluminium stearate gel or hydrogenated edible fats, emulsifying agents, for example lecithin, sorbitan, monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

It should be understood that in addition to the ingredients particularly mentioned above, the formulations may include other agents conventional in the art having regard to the type of formulation in question.

The compositions of the present invention may be suitable for the treatment of diseases in a human or animal patient. In one embodiment, the patient is a mammal including a human, horse, dog, cat, sheep, cow, or primate. In one embodiment the patient is a human. In a further embodiment, the patient is not a human.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein the term “treatment” refers to defending against or inhibiting a symptom, treating a symptom, delaying the appearance of a symptom, reducing the severity of the development of a symptom, and/or reducing the number or type of symptoms suffered by an individual, as compared to not administering a pharmaceutical composition of the invention. The term treatment encompasses the use in a palliative setting

Accordingly, the present invention, in one embodiment, relates to a method for the treatment of an infectious disease in a mammal comprising administering a therapeutically effective amount of bucillamine or a pharmaceutically acceptable salt or solvate thereof, to a mammal in need thereof. In another aspect, the infectious disease is influenza. In another aspect, the infectious disease is coronavirus disease 2019.

Clinical Trial

A Phase 3 confirmatory clinical trial titled, “A Multi-Center, Randomized, Double-Blind, Placebo-Controlled Study of Bucillamine in Patients with Mild-Moderate COVID-19”, was undertaken to enroll up to 1,000 patients randomized 1:1:1 to receive Bucillamine 100 mg three times a day (“TID”), Bucillamine 200 mg TID or placebo TID for up to 14 days. The primary objective is to compare the frequency of hospitalization or death in patients with mild-moderate COVID-19 receiving Bucillamine therapy with those receiving placebo. The primary endpoint is the proportion of patients meeting a composite endpoint of hospitalization or death from the time of the first dose through Day 28 following randomization. Efficacy was assessed by comparing clinical outcomes (death or hospitalization), disease severity using the 8-category NIAID COVID ordinal scale, supplemental oxygen use, and progression of COVID-19 between patients receiving standard-of-care plus Bucillamine (high dose and/or low dose) and patients receiving standard-of-care plus placebo. Preliminary indications are that none of the patients receiving Bucillamine in the trial have to date been hospitalised for COVID-19 or have died from COVID-19.

The present invention, in another embodiment, relates to a use of a pharmaceutical composition including bucillamine or a pharmaceutically acceptable salt or solvate thereof, together with one or more pharmaceutically acceptable carriers, diluents and excipients for the treatment of an infectious disease. In another aspect, the infectious disease is influenza. In another aspect, the infectious disease is coronavirus disease.

### REFERENCES

1 Muthuri S G, Venkatesan S, Myles P R et al. Effectiveness of neuraminidase inhibitors in reducing mortality in patients admitted to hospital with influenza A H1N1pdm09 virus infection: a meta-analysis of individual participant data Lancet Respir Med. 2014 May; 2(5):395-404. doi: 10.1016/S2213-2600(14)70041-4.
2 Duwe S. Influenza viruses—antiviral therapy and resistance. GMS Infect Dis. 2017; 5: Doc04.
3 Zhang R H, Li C H, Wang C L et al. N-acetyl-1-cystine (NAC) protects against H9N2 swine influenza virus-induced acute lung injury. Int Immunopharmacol. 2014 September; 22(1):1-8. doi: 10.1016/j.intimp.2014.06.013.
4 Ungheri D, Pisani C, Sanson G et al. Protective effect of n-acetylcysteine in a model of influenza infection in mice. Int J Immunopathol Pharmacol. 2000 September-December; 13(3):123-128.
5 De Flora S, Grassi C, and Carati L. Attenuation of influenza-like symptomatology and improvement of cell-mediated immunity with long-term N-acetylcysteine treatment. Eur Respir J 1997; 10: 1535-1541 DOI: 10.1183/09031936.97.10071535
6 Poole L B. The Basics of Thiols and Cysteines in Redox Biology and Chemistry. Free Radic Biol Med. 2015 March; 0: 148-157. doi: 10.1016/j.freeradbiomed.2014.11.013.
7 Mata M, Morcillo E, Gimeno C, Cortijo J. N-acetyl-L-cysteine (NAC) inhibit mucin synthesis and pro-inflammatory mediators in alveolar type II epithelial cells infected with influenza virus A and B and with respiratory syncytial virus (RSV). Biochem Pharmacol. 2011 Sep. 1; 82(5):548-55. doi: 10.1016/j.bcp.2011.05.014.
8. Horowitz L D. Bucillamine: a potent thiol donor with multiple clinical applications. Cardiovasc Drug Rev. 2003 Summer; 21(2):77-90.
9. Sagawa A, Fujisaku A, Ohnishi K et al. A multicentre trial of bucillamine in the treatment of early rheumatoid arthritis (SNOW study). Mod Rheumatol. 2011 June; 21(3):251-7. doi: 10.1007/s10165-010-0385-4.

1. A method for the treatment or prevention of an infectious disease in a mammal comprising administering a therapeutically effective amount of bucillamine or a pharmaceutically acceptable salt or solvate thereof to a mammal in need thereof.

2. The method of claim 1, wherein the infectious disease is coronavirus disease 2019.

3. The method of claim 2, wherein the therapeutically effective amount is 100 mg.

4. The method of claim 2, wherein the therapeutically effective amount is 200 mg.

5. The method of claim 2, wherein the therapeutically effective amount is in the range of about 100 mg to about 200 mg.

6. The method of claim 2, wherein the therapeutically effective amount is in the range of about 10 mg to about 50 mg.

7. The method of claim 2, wherein the therapeutically effective amount is 300 mg per day.

8. The method of claim 2, wherein the therapeutically effective amount is 600 mg per day.

9. The method of claim 1, wherein the infectious disease is influenza.

10. The method of claim 9, wherein the therapeutically effective amount is 100 mg.

11. The method of claim 9, wherein the therapeutically effective amount is 200 mg.

12. The method of claim 9, wherein the therapeutically effective amount is in the range of about 100 mg to about 200 mg.

13. The method of claim 9, wherein the therapeutically effective amount is in the range of about 10 mg to about 50 mg.

14. The method of claim 9, wherein the therapeutically effective amount is 300 mg per day.

15. The method of claim 9, wherein the therapeutically effective amount is 600 mg per day.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)
